(19) United States
(12) Patent Application Publication
(10) Pub. No.: US 2021/0000704 A1
(43) Pub. Date: Jan. 7, 2021

(54) COSMETIC COMPOSITIONS USING TITANIUM DIOXIDE PARTICLES FOR IR PROTECTION

(71) Applicant: Kobo Products, Inc., South Plainfield (JP)

(72) Inventors: Yun Shao, Belle Mead, NJ (US); Maitree Kanjilal, Metuchen, NJ (US)

(73) Assignee: Kobo Products, Inc., South Plainfield, NJ (US)

(21) Appl. No.: 16/621,958

(22) PCT Filed: Jun. 16, 2018
(86) PCT No.: PCT/US2018/037957

§ 371 Date: Dec. 12, 2019

Related U.S. Application Data
(60) Provisional application No. 62/521,043, filed on Jun. 16, 2017.

(51) Int. Cl. A61K 8/02; A61K 8/29; A61Q 19/00; A61Q 1/08; A61Q 17/04; A61K 8/891; A61K 8/19; A61K 8/27

(52) U.S. Cl. CPC A61K 8/0245; A61K 8/29; A61Q 19/007; A61Q 1/08; A61K 2800/26; A61K 8/891; A61K 8/19; A61K 8/27; A61Q 17/04

(57) ABSTRACT

Disclosed are personal care compositions comprising acicular titanium dioxide, which block infrared radiation in the NIR spectral range, and methods for making them.

### CROSS-REFERENCE TO RELATED APPLICATIONS

This application claims priority under 35 U.S.C. § 119(e) to U.S. Provisional Application Ser. No. 62/521,043, filed Jun. 16, 2017, the disclosure of which is incorporated herein by reference.

### FIELD OF THE INVENTION

The present invention is related to infrared attenuation and more particularly using a TiO₂ particle of a specific size and shape, and its use in personal care products, especially those for sun care compositions. The invention also includes surface modification of the TiO₂particles.

### BACKGROUND OF THE INVENTION

The public and medical community generally recognize that over exposure to the sunlight can potentially cause skin cancers and pre-mature ageing due to the presence of ultraviolet (UV) light. Solar radiation includes about 5% ultraviolet (UV) radiation with a wavelength in the range between 200 nm and 400 nm, which can be further classified into three regions: from 320 to 400 nm (UV-A), 290 to 320 nm (UV-B) and from 200 to 290 nm (UV-C). While a large part of UV-C radiation is absorbed by the ozone layer, exposure to UV-A and UV-B radiation for short period causes reddening of the skin and localized irritation, and continued and prolonged exposure can lead to sunburn, melanoma and formation of wrinkles. It is also reported that UV radiation causes significant damage to hair.

As a result, sunscreens have long been developed and used by consumers to protect against UV light. However, potential harmful effect of Infrared (IR) light irradiated by the Sun to the skin caused attention only much later. Nevertheless, in the recent decades, it has been generally recognized that IR radiation from sunlight may contribute to ageing and carcinogenesis by amplifying ultraviolet injury, altering the vasculature, producing diffusible mediators, changing histone binding properties, and/or damaging DNA repair processes. (Kaidbey, et al., Arch. Dermatol., 1982, 118(5), pp 315-318; L. Keligman, Arch Dermatol Res., 1982, 272(3-4), pp 229-238).

Similar to UV light, IR can also be classified into three regions based on wavelengths: IRA (750 nm-1,400 nm), IRB (1,400 nm to 3,000 nm), and IRC (3,000 nm-1 mm). IR with a wavelength in the range of 0.7 to 2.5 μm, which covers all IRA and most of IRB, is often called near IR (NIR). IRA rays represent about one-third of total solar energy. They are capable of penetrating human skin and directly affecting cells located in the epidermis, dermis, and subcutis. This is in contrast to the IRC (3,000 nm-1 mm) or the IRB (1,400-3,000 nm), which are completely absorbed at the epidermis or only marginally affect the dermis. IRA, similar to UVA or UVB, can cause skin damage and significantly contribute to the photoaging of human skin. (see, e.g., M. S. Kim, et al., Mech. Ageing Dev., 2006, 127:875-882; P. Schroeder, et al., Skin Pharmacol. Physiol., 2010, 23:15-17).

In fact, IRA rays penetrate skin more deeply than either UVA or UVB rays, passing through the epidermis and dermis into the subcutaneous layer. These rays are responsible for the warmth one feels on the skin exposed to the sun, but they also generate free radicals that could cause collagen breakdown and accelerate skin ageing. The heat produced by infrared rays can also lead to inflammation in the skin, which over time plays a role in premature signs of ageing. Since so much infrared energy comes into contact with one's skins every day, sunscreens that offer infrared, especially IRA, protection are highly desirable against sunburn, premature photoaging and skin cancer.

For protection against IR rays, antioxidants such as L-ascorbic acid (vitamin C), tocopherols (vitamin E), ubiquinone (coenzyme Q10), glutathione, alpha lipoic acid, betacarotein, ferulic acid, oleuropein and others have been used in the cosmetic industry to mitigate the damage to the skin. Particulates that can block the IR light have also been used. For example, U.S. Pat. No. 8,647,609 disclosed infrared ray blocking particles composed of titanium dioxide (TiO₂) or zinc oxide (ZnO) within the particle size range of 0.38-1.5 μm. U.S. Pat. No. 9,480,632 disclosed the use of inorganic powders selected from cerium oxide, talc, aluminum oxide, iron oxide, zinc oxide, and mica with an average particle size of 0.1-40 μm to protect against UVA, UVB and near-IR simultaneously. U.S. Pat. No. 5,427,771 disclosed the use of titanium dioxide flakes (0.5 to 10% by weight) having dimensions between 1.5 and 25 microns to protect against IR light. CN 1196233 disclosed health-care compositions having far infrared cosmetic features, which contain a ceramic powder of alumina, iron oxide, silicon oxide, calcium sulfate, and zinc stearate, and absorbs far infrared in a wavelength range of 5.6-15 microns with a far infrared emissivity of 85-98% for optimal absorption by skin and hypoderm.

Despite all these efforts, new materials and methods for personal care protection against solar IR radiation are still in hot pursuit with the increasing awareness about the importance of such protection by customers.

### SUMMARY OF THE INVENTION

The present invention provides a solution to the issue imposed by IR radiation, based upon a surprising discovery that titanium dioxide particles in an acicular shape have a high IR attenuation power.

In one aspect, the present invention provides a sun care composition comprising acicular TiO₂ particles and optionally another organic or inorganic UV filters.

In another aspect, the present composition provides a cosmetic or personal care formulation comprising a sunscreen composition according to any embodiments, or combinations thereof, as disclosed herein.

In another aspect, the present composition provides an article comprising a sunscreen composition according to any embodiments, or combinations thereof, as disclosed herein.

These and other aspects and advantages of the present invention will become more apparent in view of the following detailed description, drawings, and claims.

### BRIEF DESCRIPTION OF THE DRAWINGS

FIG. 1 shows an electron micrograph of acicular TiO₂ FTL-100.

FIG. 2 shows an electron micrograph of acicular TiO₂ FTL-200.

FIG. 3 shows an electron micrograph of acicular TiO₂ FTL-300.

FIG. 4 illustrates the IR transmittance curves of eight types of TiO₂ particles tested.

FIG. 5 illustrates the IR Transmittance of Most Effective Grades of TiO₂ particles.

### DETAILED DESCRIPTION OF THE INVENTION

In one aspect, the present invention provides a sun care composition for protection against IR rays in solar radiation, the composition comprising acicular, i.e., needle-shaped, TiO₂ particles.

TiO₂ has been widely used as a white pigment in part because its high refractive index leads to a very high opacity. Micronized or nano TiO₂ has been widely used as sunscreen active ingredient to attenuate harmful UV light due to their excellent ability to absorb and scatter UV light.

As a rule of thumb, TiO₂ scatters light most effectively when its size is about the half of the light wavelength. This requires the size for blocking IR light to be over 300 nm, in which range TiO₂ particles typically have a granular shape. In contrast, acicular TiO₂ is highly crystalline like needles, for example, those manufactured by Ishihara Sangyo Kaisha, Ltd. under the trade name of FTL series.

In one embodiment, the sun care composition of the present invention contains the acicular (needle-shaped) TiO₂ with a diameter of 0.1-2 μm, preferably 0.2-0.5 μm, and a length of 1-40 μm, preferably 3-10 μm.

In one embodiment, the TiO₂ is not coated. The TiO₂ can be rutile or anatase.

In another embodiment, the TiO₂ is coated with a different metal oxide compound, such as alumina or silica.

In another embodiment, the TiO₂ is additionally coated with an organic material.

In some embodiments, the organic material is selected from the group consisting of alkoxysilanes, silicones, organic titanates, fatty acids, metal soaps, polyols, and combinations of two or more thereof.

In another embodiment, the TiO₂ is additionally coated with a hydrophilic organic material.

In some embodiments, the hydrophilic organic material is selected from the group consisting of polyethylene glycol (PEG), silane, polyacrylate salt, polysaccharide, water soluble silicone polyether, and combinations of two or more thereof.

In another embodiment, preferably, the TiO₂ is used in skincare formulations, especially sun care products. It can be used in combination with common organic and/or inorganic sunscreen active ingredients. The TiO₂ can be used in cosmetic composition at a level 1-25%, but preferably 2-10%, by weight.

The sun care composition of the invention can be formulated as different cosmetic products by adding the corresponding ingredients common for such products. These products include, for example, lotions, oils, day care products with UV protection, gels, masks, balms, powders, eye-liftings, tan glows, tinted creams, pre-sun products, sun products, make-ups, compact powders, photo protecting products, sprays, blush powders, and lipsticks, or the like.

In some embodiments, the sun care composition of the present invention further comprises one or more UV filters selected from UV-A filters, UV-B filters, both UV-A and UV-B filters, and combinations thereof.

Any organic UV filters can in principle be used in combination with acicular TiO₂, including but not limited to p-aminobenzoic acid (PABA), octyldimethyl-PABA, phenylbenzimidazole sulfonic acid (PBSA), 2-ethoxyethyl p-methoxycinnamate, dioxybenzone, oxybenzone, homomethyl salicylate (HMSA), menthyl anthranilate, 2-cyano-3,3-diphenyl acrylic acid, 2-ethylhexylester (“Octocrylene”), octyl methoxycinnamate, octyl salicylate, 2-hydroxy-4-methoxybenzophenone-5-sulfonic acid, triethanolamine salicylate, butyl methoxy dibenzoylmethane (BMBDM), terephthalylidene dicamphor sulfonic acid, 4-methylbenzylidene camphor (MBC), methylene bis-benzotriazolyl tetramethylbutylphenol (MBBT), bis-ethylhexyloxyphenol methoxyphenol triazine (BEMT), tris-biphenyl triazine, disodium phenyl dibenzimidazole tetrasulfonate (DPDT), drometrizole trisiloxane, sodium dihydroxy dimethoxy disulfobenzophenone, ethylhexyl triazone (EHT), diethylamine hydroxybenzoyl hexyl benzoate, diethylhexyl butamido triazone (DBT), dimethico-diethylbenzalmalonate, and isoamyl p-methoxycinnamate (IMC), and combinations thereof.

Inorganic UV filters include, but are not limited to, zinc oxide, titanium dioxide particulates (granular, spherical, etc.), iron oxides, kaolin, talc, phosphate salts, carbonate salts, hydroxyapatite, zinc sulphide, cadmium yellow, bismuth vanadate, and combinations thereof.

In some embodiments, the UV filters are added to the sunscare composition so that they have additive effects to the effect of acicular TiO₂ blocking IRA.

In some preferred embodiments, UV filters are added to the sun care composition so that they have synergistic effects on the effect of acicular TiO₂ blocking IRA, that is, both the blocking effects against UV and against IRA are enhanced.

Therefore, any existing suitable sunscreen formulation for UV protection may be a suitable “substrate” for preparing a sunscreen formulation of the present invention by adding an acicular TiO₂ material; provided, however, that the new formulation produced is stable for storage and use. Therefore, sunscreen formulations thus formed will be protective against both UV and IRA radiations.

In some embodiments, the sunscreen formulations of the present invention can be formulated specifically for protection against IR radiation, and used in combination with a regular sunscreen composition separately.

Emulsion products suitable for the present invention include multiple emulsions, micro emulsions, and nano emulsions in the form of W/O, O/W, W/Si, Si/W, W/O/W, O/W/O, O/W/Si, and W/Si/W emulsions (where O=Oil, W=Water, Si=Silicone). Other products, such as anhydrous systems like Si/O, are also included.

The sunscreen products can be in a variety of forms, including but not limited to gels, creams, lotions, oils, sprays, or daily protective skin care products with different Sun Protection Factors (SPF) in the range from SPF 2 to SPF 50+, e.g., SPF 6, SPF 10, SPF 15, SPF 20, SPF 25, SPF 30, SPF 50, and SPF 50+. The different SPFs are dependent on the kind and amount of UV filter substances.

The term “sun care composition” or “sunscreen composition”, as used herein, refers to a composition for topical application to skin and/or hair of mammals, especially humans, for sunscreen benefits. Such a composition may be generally classified as leave-on or rinse off, and includes any product applied to a human body primarily for sun protection but may be used also for improving appearance, cleansing, odor control, or general aesthetics.

“Skin” as used herein is meant to include skin on the face and body (e.g., neck, chest, back, arms, underarms, hands, legs, buttocks and scalp) and especially to the sun exposed parts thereof. The sun care composition of the invention is also of relevance to applications on any other keratinous substrates of the human body other than skin e.g. hair where products may be formulated with specific aim of providing photoprotection.

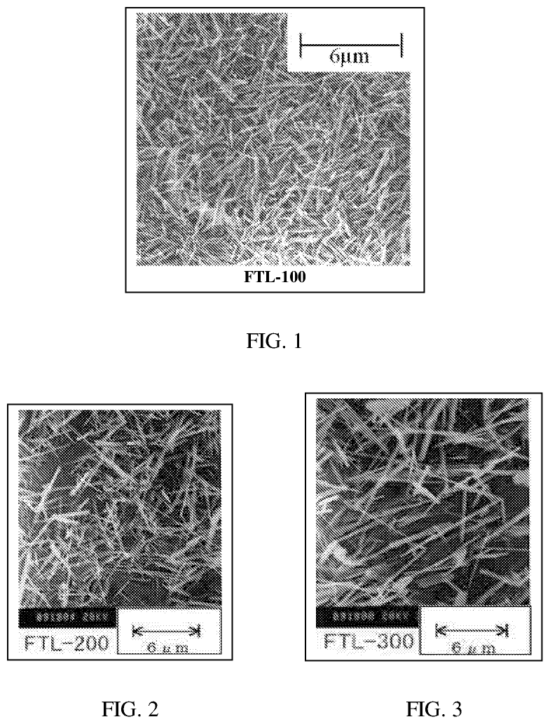

The structures of this type of acicular TiO₂ can be viewed through electronic micrograph. See FIGS. 1 to 3. These acicular TiO₂ particles were compared with other TiO₂ particles having typical sizes and shapes on the market, with their selected properties listed in Table 1 below.

TABLE 1
Physical properties of various types of TiO₂ particles.

| Trade name | Composition | PPS (μm) | Shape |
|---|---|---|---|
| MT-100WP | TiO₂ coated with silica | 0.014 × 80 | Acicular |
| AFDC-200 | TiO₂ | 0.17 | Granular |
| ST-730EC | TiO₂ coated with Al₂O₃ | 0.5 | Granular |
| | and silicone | | |
| MP-100 | TiO₂ | 1 | Granular |
| ST-750EC | TiO₂ coated with Al₂O₃ | 1 | Granular |
| | and silicone | | |
| HERITITAN | TiO₂ | 2-7 | Spherical |
| AA-1514 | | | |
| FTL-100 | TiO₂ | 0.13 × 1.7 | Acicular |
| FTL-300 | TiO₂ | 0.27 × 5.2 | Acicular |

The IR attenuation power of these TiO₂ particles was tested using Perkin Elmer 400 FT-IR/FT-NIR Spectrometer according to method described below.

### I. Sample Preparation

A: Slurry preparation
1. Samples were weighed and added to SF96® 1000 Silicone Fluids at the desired percentages and dispersed on Speedmixer® for 30 seconds at 2000 rpm.
2. Samples were added to an emulsion at the desired percentages and dispersed on Speedmixer® for 30 seconds at 2000 rpm.
   B: Drawdown preparation
1. The slurry was applied to a fused IR Quartz Window (50 mm diameter×3 mm thickness) and drawn down using 0.5 mil (13 microns) wire rod.
2. The drawdown was air-dried for 5 min

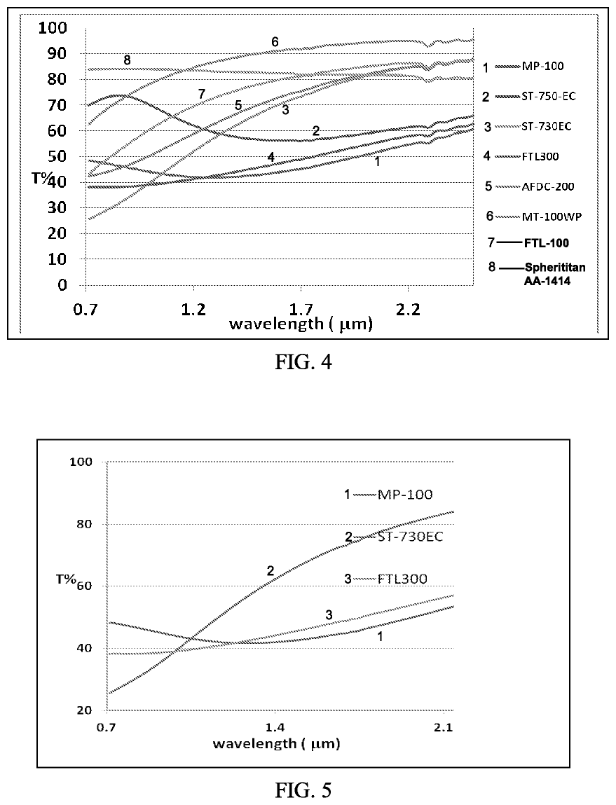

Test Conditions: 64 cm⁻¹ resolution, scan range 14,286-4,000 cm⁻¹ (0.7-2.5 μm). Transmittance spectra in the NIR spectral range (0.7-2.5 μm) were collected and compared. FIG. 4 includes the overlay of transmittance spectra of eight different TiO₂ materials, and FIG. 5 includes transmittance spectra of three most effective grade of TiO₂ materials in blocking NIR.

The transmittance curves of FIG. 4 and FIG. 5 clearly demonstrate that, with respect to blocking NIR radiation:
1. Nano TiO₂ is not effective, perhaps because the particles are too small to be able to scatter the IR rays effectively.
2. As the particle size increases, the attenuation of IR rays increases. The commonly used pigmentary TiO₂ particles were observed to have some IR attenuation power.
3. Granular TiO₂ particles with a size of 0.5-1 micron were observed to have high IR attenuation power.
4. It was surprisingly observed that the acicular TiO₂, in particular, FLT-300, showed a higher attenuation power than any other grades of TiO₂. It has a similar efficiency in blocking IR rays to that of MP-100, 1 micron TiO₂ from Tayca Corporation. FLT-300 is most effective to block IR rays in the wavelength range of 0.7-1.25 μm, a range of IR light with highest energy and most damaging effects, which makes this acicular TiO₂ particularly protective against IR.

### EXAMPLES

The following non-limiting formulation examples are provided to further illustrate certain aspects of the present invention.

### Example 1

### Formula 1. Moisturizing Skin Lotion

| Part | % W/W | Ingredient | INCI names |
|---|---|---|---|
| 1 | 61.38 | Deionized Water | Water |
| | 5 | FTL-300 acicular | Titanium dioxide |
| | | TiO₂ | |
| 2 | 7.62 | Glycerin | Glycerin |
| | 0.48 | Keltrol ® CG-T | XANTHAN GUM |
| 3 | 9.52 | Lexfeel 7 | Neopentyl Glycol |
| | | | Diheptanoate |
| | 4.76 | Protachem CTG | Caprylic/Capric |
| | | | Triglyceride |
| | 2.86 | Salacos 99 | Isononyl isononanoate |
| | 1.43 | Lanette ® O | Cetearyl Alcohol |
| | 1.9 | Dermofat 4919 | Stearic acid |
| | 1.9 | Polyaldo 10-1-0 K FG | Polyglyceryl-10 Oleate |
| | 0.86 | Phenoxyethanol | Phenoxyethanol |
| 4 | 1.9 | Glycerine | Glycerine |
| | 0.38 | AQUA KEEP 10SH-NFC | Sodium Acrylates |
| | | | Crosspolymer-2 |
| | 100 | | |

Preparation procedure:
1. Combined Part 3 and heated to 80° C.
2. Mixed Part 1 using a propeller mixer, then added Part 2. Heated the mixture to 80° C.
3. Added Part 3 to Part 1 and Part 2 and kept mixing for 15 min
4. Began cooling batch to 45° C.
5. Cooled to 25° C.

### Example 2

### Formula 2. Foundation

| Part | % W/W | Ingredient | INCI Name |
|---|---|---|---|
| 1 | 3.56 | Jeechem CTG | Caprylic/Capric Triglyceride |
| | 2.86 | Dermofat 4919 | Stearic Acid |
| | 1.43 | Lipo ® GMS-450 | Glyceryl Stearate |
| | 0.48 | Lipocol ® C | Cetyl Alcohol |
| 2 | 11.90 | GCB60USG | Titanium Dioxide (And) Caprylic/ |
| | | | Capric Triglyceride (And) |
| | | | Isopropyl Myristate (And) |
| | | | Stearoyl Glutamic Acid (And) |
| | | | Stearalkonium Hectorite (And) |
| | | | Trihydroxystearin (And) |
| | | | Propylene Carbonate |
| | 1.90 | GCB50YSG | Iron Oxides (CI 77492) |
| | 0.45 | GCB65RSG | Iron Oxides (CI 77491) |
| | 0.28 | GCB70BSG | Iron Oxides (CI 77499) |
| | 5.00 | FTL-300 acicular | Titanium dioxide and isopropyl |
| | | TiO₂ with coating | titanium triiso stearate |
| 3 | 65.90 | Deionized Water | Water |
| | 4.76 | Butylene Glycol | Butylene Glycol |
| | 0.95 | Triethanolamine 99 | Triethanolamine |
| | 0.24 | Keltrol ® CG | Xanthan Gum |
| | 0.19 | Germall ® 115 | Imidazolidinyl Urea |
| | 0.10 | Methyl Paraben NF | Methylparaben |
| Total | 100 | | |

Preparation procedure:
p 1. Combined Part 1 and Part 2 under propeller mixing and heated to 80° C. until color became uniform.
2. Began propeller mixing water in Part 3 at 80° C. Then added remaining ingredients with butylene glycol and xanthan gum as slurry until batch became uniform.
3. Slowly added Part 3 to Part 1 and Part 2 under propeller mixing until uniform.
4. Began cooling batch until 65° C.

### Example 3

### Formula 3. SPF 30 Sunscreen Lotion

| Part | % W/W | Ingredient |
|---|---|---|
| 1 | 56.29 | Deionized Water |
| | 1.00 | Polysorbate 20 |
| 2 | 1.50 | Propylene Glycol |
| | 0.30 | Xanthan Gum |
| | 5.00 | FTL-300 acicular TiO₂ |
| 3 | 11.36 | TNP65FZS - 65% ZnO Dispersion in C12-15 |
| | | alkylbenzoate |
| 4 | 6.00 | Homosalate |
| | 7.50 | Octyl Methoxcinnamate |
| | 2.00 | Octocrylene |
| | 5.00 | Glyceryl Stearate |
| | 0.75 | potassium cetyl phosphate (Amphisol K) |
| | 0.50 | Mixed Tocopherols |
| | 0.50 | Cetearyl Alcohol (and) Ceteareth-20 |
| | 0.30 | Sorbitan Oleate |
| 5 | 1.00 | Phenoxyethanol (and) |
| | | Caprylyl Glycol (and) |
| | | Sorbic Acid |
| | | (Optiphen Plus) |
| 6 | 1.00 | EG-150/Decyl Alcohol/SMDI |
| | | Copolymer (Aculyn 44) |
| Total | 100.00 | |

Preparation procedure:
1. Combined Part 1 and heated to 70° C.
2. Combined Part 2 ingredients into a slurry, and added to Part 1 while mixing with a propeller mixer.
3. Combined Part 3 and Part 4, and heated to 70° C.
4. Added the mixture of Part 3 and Part 4 to the mixture of Part 1 and Part 2 under homogenization.
5. Added Part 5 under propeller mixing while cooling.
6. Cooled to 40° C., then added Part 6 under propeller mixing.

### Example 4

### Formula 4. SPF 50 Sunscreen Lotion

| Part | % W/W | Ingredient |
|---|---|---|
| 1 | 49.66 | Water |
| | 5.00 | acicular titanium dioxide (FTL-300) |
| | 2.86 | Propylene Glycol |
| | 0.48 | Sodium Chloride |
| | 0.19 | Allantoin |
| | 0.14 | Methylparaben |
| 2 | 9.52 | Octocrylene |
| | 9.52 | C12-15 Alkyl Benzoate (And) |
| | | Titanium Dioxide (And) |
| | | Alumina (And) |
| | | Polyhydroxystearic Acid (And) |
| | | Isopropyl Titanium Triisostearate (And) |
| | | Triethoxycaprylylsilane (TNP40VTTS) |
| | 8.10 | Ethylhexyl Methoxycinnamate |
| | 3.81 | Ethylhexyl Stearate |
| | 2.38 | PEG-30 Dipolyhydroxystearate |
| | 2.38 | Polyglyceryl-4 Isostearate (And) |
| | | Cetyl PEG/PPG-10/1 Dimethicone (And) |
| | | Hexyl Laurate (ABIL ® WE 09) |
| | 1.43 | Benzophenone-3 |
| | 0.95 | Cetyl Dimethicone |
| | 0.95 | Cyclopentasiloxane (And) Cyclohexasiloxane |
| | 0.95 | Cetearyl Alcohol (And) |
| | | Dicetyl Phosphate (And) |
| | | Ceteth-10 Phosphate |
| | 0.95 | Shea Butter |
| | 0.48 | Microcrystalline Wax |
| | 0.19 | Rosemarinus Officinalis (Rosemary) |
| | | Leaf Extract |
| | 0.06 | Propylparaben |
| Total | 100 | |

Preparation procedure:
1. In a double jacketed stainless steel tank equipped with a lightning type mixer along with side sweep action, mixed Part 1 in order listed and heated to 70° C.
2. In a second double jacketed stainless steel tank heated Part 2 to 70° C.
3. Added Part 1 to Part 2 slowly with mixing using a lightning type mixer.
4. Switched the mixer to side sweep mixing and started to cool.

5. Continued to cool and to mix emulsion until the temperature dropped below 35° C.

It will be understood by those of skill in the art that numerous and various modifications can be made without departing from the spirit or scope of the present invention. Therefore, the various embodiments of the present invention described herein are illustrative only and not intended to limit the scope of the present invention. All patent or non-patent references cited herein are incorporated by reference in their entirety, and citation of them does not constitute admission or otherwise acknowledgement of them as prior art.

1. A personal care composition comprising acicular TiO₂ particles having an average diameter in the range of about 0.1-2 μm and an average of length in the range of 1-40 μm and capable of blocking near infrared radiation in the wavelength range of 0.7 to 2.5 μm.

2. The personal care composition of claim 1, wherein the acicular TiO₂ particles are non-coated rutile or anatase.

3. The personal care composition of claim 1, wherein the acicular TiO₂ particles are coated with an oxide coating material.

4. The personal care composition of claim 3, wherein the oxide coating material comprises silica, alumina, or a mixture thereof.

5. The personal care composition of claim 3, wherein the TiO₂ particles are additionally coated with an organic coating material.

6. The personal care composition of claim 5, wherein the organic coating material is selected from the group consisting of silanes, reactive methicones, dimethicones, branched dimethicones, organic titanates, fatty acids, metal soaps, polyols, and combinations of two or more thereof.

7. The personal care composition of claim 5, wherein the organic coating material is a hydrophilic organic material selected from the group consisting of PEG ether silanes, polyacrylate salts, polysaccharides, water soluble silicone polyethers, and combinations of two or more thereof.

8. The personal care composition of claim 1, wherein the acicular TiO₂ is present at a level from about 1% to 25% by weight of the total composition.

9. The personal care composition of claim 1, wherein the acicular TiO₂ is introduced into the composition in the form of dispersion.

10. The personal care composition of claim 1, wherein the composition is unpigmented for skin, hair, or nail care.

11. The personal care composition of claim 1, wherein the composition is a color cosmetic selected from the group consisting of foundations, lipsticks, loose powders, nail polish, and pressed powders.

12. The personal care composition of claim 1, further comprising one or more additional organic and/or inorganic UV filters.

13. The personal care composition of 12, wherein the additional organic and/or inorganic UV filters are selected from the group consisting of p-aminobenzoic acid (PABA), octyldimethyl-PABA, phenylbenzimidazole sulfonic acid (PBSA), 2-ethoxyethyl p-methoxycinnamate, dioxybenzone, oxybenzone, homomethyl salicylate (HMSA), menthyl anthranilate, 2-cyano-3,3-diphenyl acrylic acid, 2-ethylhexylester (“Octocrylene”), octyl methoxycinnamate, octyl salicylate, 2-hydroxy-4-methoxybenzophenone-5-sulfonic acid, triethanolamine salicylate, butyl methoxy dibenzoylmethane (BMBDM), terephthalylidene dicamphor sulfonic acid, 4-methylbenzylidene camphor (MBC), methylene bis-benzotriazolyl tetramethylbutylphenol (MBBT), bis-ethylhexyloxyphenol methoxyphenol triazine (BEMT), tris-biphenyl triazine, disodium phenyl dibenzimidazole tetrasulfonate (DPDT), drometrizole trisiloxane, sodium dihydroxy dimethoxy disulfobenzophenone, ethylhexyl triazone (EHT), diethylamino hydroxybenzoyl hexyl benzoate, diethylhexyl butamido triazone (DBT), dimethico-diethylbenzalmalonate, isoamyl p-methoxycinnamate (IMC), zinc oxide, non-acicular titanium dioxide particulates, iron oxides, kaolin, ichthammol, talc, calamineinm, phosphate salts, carbonate salts, hydroxyapatite, zinc sulphide, cadmium yellow, bismuth vanadate, and combinations thereof.

14. A personal care composition selected from the group consisting of:
(a) a moisturizing skin lotion comprising acicular titanium dioxide, glycerin, xanthan gum, neopentyl glycol diheptanoate, caprylic/capric triglyceride, isononyl isononanoate, cetearyl alcohol, stearic acid, polyglyceryl-10 oleate, phenoxyethanol, glycerine, sodium acrylates crosspolymer;
(b) a foundation formulation comprising acicular titanium dioxide, caprylic/capric triglyceride, stearic acid, glyceryl stearate, cetyl alcohol, isopropyl myristate, stearoyl glutamic acid, stearalkonium hectorite, trihydroxystearin, propylene carbonate, iron oxides, isopropyl titanium triisostearate, butylene glycol, triethanolamine, xanthan gum, imidazolidinyl urea, and methylparaben;
(c) an SPF 30 sunscreen lotion comprising acicular titanium dioxide, polysorbate 20, propylene glycol, xanthan gum, 65% ZnO dispersion in C₁₂₋₁₅ alkyl benzoate, homosalate, octyl methoxcinnamate, octocrylene, glyceryl stearate, potassium cetyl phosphate, mixed tocopherols, cetearyl alcohol, Ceteareth-20, sorbitan oleate, phenoxyethanol, caprylyl glycol, sorbic acid, (Optiphen plus), EG-150/decyl alcohol/SMDI copolymer (Aculyn 44); and
(d) an SPF 50 sunscreen lotion comprising acicular titanium dioxide, propylene glycol, sodium chloride, allantoin, methylparaben, octocrylene, C₁₂₋₁₅ alkyl benzoate, alumina, polyhydroxystearic acid, isopropyl titanium triisostearate, triethoxycaprylylsilane, ethylhexyl methoxycinnamate, ethylhexyl stearate, PEG-30 dipolyhydroxystearate, polyglyceryl-4 isostearate, cetyl PEG/PPG-10/1 dimethicone, hexyl laurate, benzophenone-3, cetyl dimethicone, cyclopentasiloxane, cyclohexasiloxane, cetearyl alcohol, dicetyl phosphate, Ceteth-10 phosphate, shea butter, microcrystalline wax, rosemarinus officinalis (rosemary) leaf extract, and propylparaben.

15. The personal care composition of claim 4, wherein the TiO₂ particles are additionally coated with an organic coating material.

16. The personal care composition of claim 15, wherein the organic coating material is selected from the group consisting of silanes, reactive methicones, dimethicones, branched dimethicones, organic titanates, fatty acids, metal soaps, polyols, PEG ether silanes, polyacrylate salts, polysaccharides, water soluble silicone polyethers, and combinations of two or more thereof.

17. The personal care composition of claim 15, wherein the acicular TiO₂ is present at a level from about 1% to 25% by weight of the total composition.

18. The personal care composition of claim 17, which is a formulation selected from the group consisting of lotions, oils, day care products with UV protection, gels, masks, balms, powders, eye-liftings, tan glows, tinted creams, pre-sun products, sun products, make-ups, compact powders, photo protecting products, sprays, blush powders, and lipsticks.

19. The personal care composition of claim 16, wherein the acicular TiO₂ is present at a level from about 2% to 10% by weight of the total composition.

20. The personal care composition of claim 19, which is a formulation selected from the group consisting of skin care formulations, hair care formulations, nail care formulations, foundations, lipsticks, loose powders, nail polish, and pressed powders.
